(19) United States
(12) Patent Application Publication
(10) Pub. No.: US 2003/0083539 A1
(43) Pub. Date: May 1, 2003

(54) Intra-aortic balloon catheter for long-term implantation

(76) Inventor: Boris Leschinsky, Waldwick, NJ (US)

Correspondence Address: Datascope Corp., 14 Philips Parkway, Montvale, NJ, 07645, US

(21) Appl. No.: 10/313,873

(22) Filed: Dec. 6, 2002

Related U.S. Application Data
(63) Continuation-in-part of application No. 09/716,009, filed on Nov. 17, 2000.

(51) Int. Cl. A61N 1/362

(52) U.S. Cl. 600/18

(57) ABSTRACT

An intra-aortic balloon catheter system, designed for long-term use, comprising an intra-aortic balloon catheter and a sheath. The sheath comprises a felt sleeve which promotes tissue ingrowth and behaves as an infection barrier. In one embodiment of the invention, the sheath resides only in the subcutaneous tissue, and therefore, does not enter or contact the artery of the patient.

### RELATED APPLICATIONS

This is a continuation-in-part of application Ser. No. 09/716,009, filed on Nov. 11, 2000.

### BACKGROUND OF THE INVENTION

1. Field of the Invention

The invention relates to an intra-aortic balloon catheter for long-term use. More particularly, the invention relates to an intra-aortic balloon catheter, implantable in the upper body or lower body of a patient, which minimizes the risk of infection and blood flow obstruction caused by the presence of the catheter.

2. Description of the Prior Art

Intra-aortic balloon (IAB) catheters are used in patients with left heart failure to augment the pumping action of the heart. The catheters, approximately 1 meter long, have an inflatable and deflatable balloon at the distal end. The catheter is typically inserted into the femoral artery and moved up the descending thoracic aorta until the distal tip of the balloon is positioned just below or distal to the left subclavian artery. The proximal end of the catheter remains outside of the patient's body and is connected to a Y-fitting. A passageway for inflating and deflating the balloon extends through the catheter and is connected via the Y-fitting to an external pump. The patient's central aortic pressure is used to time the balloon and the patient's ECG may be used to trigger balloon inflation in synchronous counterpulsation to the patient's heart beat.

Intra-aortic balloon therapy increases coronary artery perfusion, decreases the workload of the left ventricle, and allows healing of the injured myocardium. Ideally, the balloon should be inflating immediately after the aortic valve closes and deflating just prior to the onset of systole. When properly coordinated, the inflation of the balloon raises the patient's diastolic pressure, increasing the oxygen supply to the myocardium; and balloon deflation just prior to the onset of systole lowers the patient's diastolic pressure, reducing myocardial oxygen demand.

Intra-aortic balloon catheters may also have a passageway or lumen, which can be used to measure aortic pressure. In this dual lumen construction, the central lumen may also be used to accommodate a guide wire to facilitate placement of the catheter and to infuse fluids, or to do blood sampling.

Typical dual lumen intra-aortic balloon catheters have an outer, flexible, plastic tube, which serves as the inflating and deflating gas passageway, and a central tube therethrough formed of plastic tubing, stainless steel tubing, or wire coil embedded in plastic tubing. A polyurethane compound is used to form the balloon. Other dual lumen intra-aortic balloon catheters have their central tube embedded or affixed to the inner surface of the outer tube. The inner or central tube in some intra-aortic balloon catheters may be adhered to or integrally formed with the outer tube in what is typically referred to as a co-lumen arrangement.

Intra-aortic balloon (IAB) therapy is a recognized form of temporary cardiac assist. For most patients, 1-5 days of IAB support is sufficient to assist in heart recovery. However, there are a number of patients that require a longer duration of cardiac assist. One such category of patients comprises those currently waiting on heart transplantations. Unfortunately, several months of cardiac support may be required before a donor heart becomes available. Presently, various blood pumps, rather than IAB catheters, are being used for patients requiring greater than five days of cardiac assist. However, not all of these patients require the high level of support provided by a blood pump and can be maintained by a long-term IAB catheter. A major advantage of the IAB catheter over the blood pump is that an IAB catheter can be inserted percutaneously, and therefore, obviates the need for the extensive open heart surgery associated with blood pump implantation.

Despite the above mentioned advantage of IAB catheters over blood pumps, an IAB catheter capable of being used for an extended period of time has yet to emerge in the market. This absence can be at least partially explained by the design concerns related to the long-term use of an IAB catheter. One overriding concern with long-term IAB catheter use is the risk of infection. The area where the catheter exits the skin of the patient is particularly sensitive to infections or irritation because it is essentially an open wound. Catheter infection, defined as the entrance of microorganisms at the puncture site around the catheter, is a serious complication which limits the long-term use of an IAB catheter. Microorganisms invariably gain access to the tract of the sheath and the catheter at the skin surface and grow inwards toward the sheath tract and the catheter tract to reach the vascular system. The longer the duration of IAB therapy the greater the risk of infection.

Ventricular Assist Devices (VAD) and artificial heart devices with pneumatic drive lines are also known to have an infection risk associated with long-term access of the drive tubes into the body. Several access port devices are described in the literature that attempt to address this problem. See for example, Long-Term Brachial Artery Catherization: Ischemic Complications, by Kevin T. Moran, in the Journal of Vascular Surgery (1988), 8:76-78. Various collars of felt or other graft type material have been placed around the drive tubes of these devices at the skin entrance. These collars work as an infection barrier by promoting tissue ingrowth around the tube. Tissue ingrowth around the tube assists in preventing infection from entering the body, and thus, promotion of such ingrowth is viewed as beneficial for long-term implants.

U.S. Pat. No. 4,936,826 by Disamodha discloses a combination device for long-term intravenous therapy comprising an outer sheath and an inner catheter. A synthetic Dacron (trademark) cuff is attached to the sheath near the distal end as an infection barrier. This cuff provides a surface which will seal through the growth of skin.

Although the above mentioned cuff and the felt collars used with VAD type devices behave as infection barriers the heightened danger of infection associated with long-term access of tubes into the body is still present because any microorganisms which surpass the infection barrier are provided with a pathway along the tube directly into the vasculature system or close to it.

A second concern with long-term IAB use involves implantation location. Currently, IAB catheters are inserted into the femoral artery. Standard femoral implantation, although possible, is not desirable for long-term IAB use because it requires the patient to remain bed bound for the duration of the IAB use, which for long-term use may be up to several months. Upper body implantation, on the other hand, is more appropriate for long-term use because it allows the patient to move freely and also reduces the risk of infection by moving the entry point away from the groin area. Unfortunately, due to the size of the standard IAB catheter, implantation into the upper body is not possible for most patients without compromising upper limb circulation.

Potential upper body insertion points for a long-term IAB include, but are not limited to, the subclavian, axillary, radial, and brachial arteries, as well as direct implantation into the aorta. Literature analysis indicates that 5,6, and 7 French sheaths and catheters are routinely being implanted for various therapies in these arteries, mostly for short-term duration but some for mid-term duration, e.g. a chemotherapy delivery catheter for cancer patients. Techniques are developed for percutaneous and cut-down placement of these catheters from either the left or right side of the patient.

A third concern with long-term IAB use involves the presence of the insertion sheath in the artery. The presence of the sheath in the artery, adjacent to the entry point, causes an obstruction of blood flow, which may cause complications if endured for the duration of a long-term therapy.

### SUMMARY OF THE INVENTION

Accordingly, it is an object of the invention to produce an IAB catheter system tailored for long-term use.

It is another object of the invention to produce an IAB catheter system that minimizes the risk of infection to the patient.

It is a further object of the invention to produce an IAB catheter system capable of being implanted in the upper body without significantly compromising upper limb circulation.

It is a still further object of the invention to produce a long-term IAB catheter system comprising a sheath which does not obstruct blood flow in the artery.

The invention is an intra-aortic balloon catheter system, designed for long-term use, comprising an intra-aortic balloon catheter and a sheath. The sheath comprises a felt sleeve which promotes tissue ingrowth and behaves as an infection barrier. In one embodiment of the invention, the sheath resides only in the subcutaneous tissue, and therefore, does not enter or contact the artery of the patient.

To the accomplishment of the above and related objects the invention may be embodied in the form illustrated in the accompanying drawings. Attention is called to the fact, however, that the drawings are illustrative only. Variations are contemplated as being part of the invention, limited only by the scope of the claims.

### BRIEF DESCRIPTION OF THE DRAWINGS

In the drawings, like elements are depicted by like reference numerals. The drawings are briefly described as follows.

FIG. 1 is a plan view of a long-term IAB catheter having a sheath with an infection barrier.

FIG. 2 is a plan view of a long-term IAB catheter having a mini sheath, which does not enter the artery, with an infection barrier.

FIG. 3 is a longitudinal cross sectional view of a guide wire inserted into a blood vessel of a patient.

FIG. 4 is a longitudinal cross sectional view of a guide wire and the sheath of the present invention inserted into a blood vessel of a patient.

FIG. 5 is a longitudinal cross sectional view of a dual lumen intra-aortic balloon catheter inserted over a guide wire through the sheath of the present invention into a blood vessel of a patient.

FIG. 6 is a longitudinal cross sectional view of a single lumen intra-aortic balloon catheter inserted through the sheath of the present invention into a blood vessel of a patient.

### DETAILED DESCRIPTION OF THE PREFERRED EMBODIMENTS

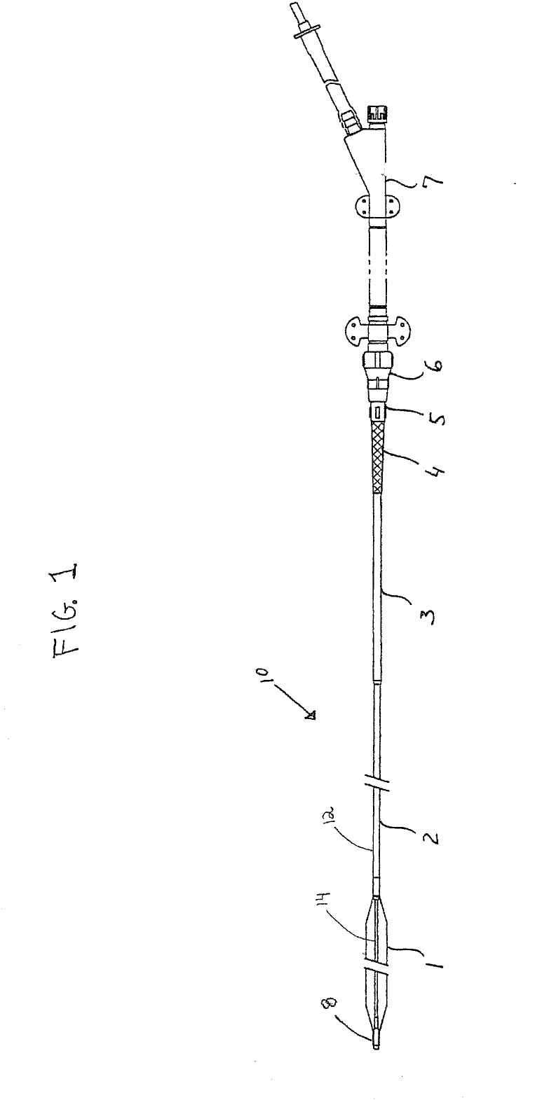

FIG. 1 illustrates a long-term IAB catheter system, designated generally as 10, comprising generally a balloon membrane 1, a catheter 2, a sheath 3, a tip 8, and a Y-fitting 7, each having proximal and distal ends. For purposes of this discussion, the term proximal refers to a portion of the long-term IAB catheter system 10 closer to the patient's heart when inserted. The distal end of the tip 8 is attached to the proximal end of the balloon membrane 1. The proximal end of the catheter 2 is attached to the distal end of the balloon membrane 1. Furthermore, the distal end of the catheter 2 is attached to the proximal end of the Y-fitting 7. Catheter 2 comprises an outer tube 12 and an optional inner tube 14 disposed within an outer surface of outer tube 12. Inner tube 14 may be coaxial with outer tube 12, as illustrated in FIG. 1, or may be connected to or integrally formed with outer tube 12; see U.S. Pat. 6,024,693, herein incorporated by reference. The sheath 3 is slidingly disposed about outer tube 12 between the balloon membrane 1 and the Y-fitting 7 and has a hub 5 and an optional Touhy-Borst type fitting 6 on its distal end. The sheath 3 also has an infection barrier sleeve 4 disposed about a portion of its distal end. The infection barrier sleeve 4 may be made from any material that behaves as an infection barrier itself or promotes tissue ingrowth, such as felt, cotton, cellulose or polymer fiber mesh. The balloon membrane 1 should have a wall thickness of less than approximately 0.005 inches (0.127 mm) and is preferably made from polyurethane.

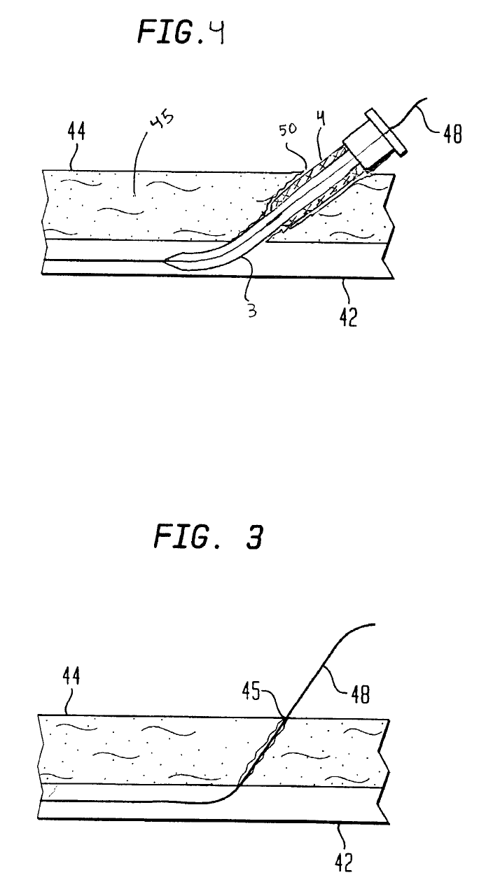

The dual lumen intra-aortic balloon catheter 2 is percutaneously inserted into an appropriate blood vessel in a manner well known in the art. First, a single incision is made in a patient 44 close to a blood vessel 42. An angiographic needle is then passed through newly created insertion site 50 and subcutaneous tissue 45 into blood vessel 42. Next, a guide wire 48 is passed through the needle into blood vessel 42. The needle is then removed leaving guide wire 48 in blood vessel 42 to guide balloon catheter 2 into blood vessel 42. See FIG. 3, which illustrates the state of affairs after the needle is removed. Alternatively, a pre-inserted guide wire from a previous procedure may be used. Guide wire 48 facilitates percutaneous insertion by guiding balloon catheter 2 through insertion site 50 and into blood vessel 42. A sheath/dilator assembly, well known in the art, is passed over the guide wire. The dilator is used to dilate tissue adjacent insertion site 50 and is then removed while leaving sheath 3 in place with a proximal end in the blood vessel and a distal end outside the patient. Sheath 3, approximately 6 inches (15.2 cm) long and having an inner diameter slightly larger than balloon catheter 2, is inserted such that the proximal end is in blood vessel 42 and infection barrier sleeve 4, a feature of the present invention, resides in subcutaneous tissue 45 only. FIG. 4 illustrates the state of affairs after the dilator has been removed, leaving sheath 50 disposed over guide wire 48 in blood vessel 42 and subcutaneous tissue 45. Note that infection barrier sleeve 4 does not enter, and preferably does not contact, blood vessel 42.

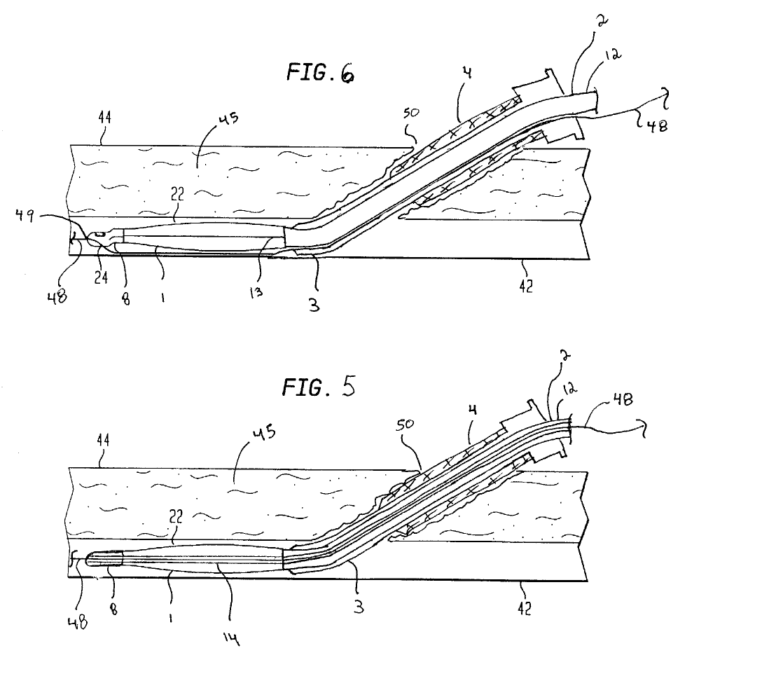

FIG. 5 illustrates the state of affairs after dual lumen intra-aortic balloon catheter 2 is advanced into blood vessel 42. Balloon catheter 2 is inserted into blood vessel 42 by passing inner tube 14 is passed over guide wire 48. It is preferred that the infection barrier sleeve 4 be made from felt and disposed about the entire length of the portion of the sheath 3 residing in the subcutaneous tissue. In a percutaneous procedure, as described above, this amounts to a length between approximately one half inch to one inch. After insertion into blood vessel 42, intra-aortic balloon catheter 2 is advanced up the descending thoracic aorta to a position appropriate for pumping: tip 8 is positioned just below or distal to the left subclavian artery.

As indicated above, balloon catheter 2 may also be a single lumen catheter, i.e. no inner tube 14, as illustrated inserted in blood vessel 42 in FIG. 6. Given the lack of an inner tube single lumen balloon catheter 2 is advanced into blood vessel 42 by passing guide wire 48 through a lumen 49 in tip 8. Similar to the dual lumen catheter in FIG. 5, after insertion into blood vessel 42, balloon catheter 2 is advanced up the descending thoracic aorta to a position appropriate for pumping: tip 8 is positioned just below or distal to the left subclavian artery.

Lumen 49 may be a self sealing lumen, as disclosed in U.S. Pat. No. 6,146,372, herein incorporated by reference in its entirety. Tip 8 may be made from a biocompatible polymer, such as polyurethane. A removable pull tube may be disposed within lumen 49 prior to insertion into blood vessel 42 to prevent occlusion of lumen 49. A pressure sensor 51 maybe connected to tip 8 to measure pressure in blood vessel 42. See U.S. patent application Ser. Nos. 09/735,076, 09/734,755 and 09/925,143, filed on Dec. 11, 2000, Dec. 11, 2000 and Aug. 9, 2001, respectively, herein incorporated by reference in their entirety, for details concerning the various means for connecting pressure sensor 51 to tip 8 and for details regarding the alternate constructions and embodiments of balloon catheter 2. Pressure sensor 51 may be a pressure transducer, or more preferably, a fiberoptic sensor. A fiber or wire (not shown) connects pressure sensor 51 to a unit for signal pickup and processing. Note that pressure sensor 51 may also be connected to dual lumen catheter 2 in FIG. 5. A stylet 13 is connected on one end to tip 8 and on an opposite end to outer tube 12.

An alternate method for inserting catheter 2, requiring at least two incisions, involves a surgical procedure called “tunneling”. First, the blood vessel is located via a cut down procedure. Next, tissue adjacent the blood vessel entry site of catheter 2 is cut so as to allow sheath 3 to tunnel through the subcutaneous tissue under the skin. Tunneling allows sheath 3 to exit through the skin at a predetermined radial distance, minimum approximately 2 inches (50.8 mm), away from a skin-level point directly above the blood vessel insertion site. The distance between the blood vessel insertion site and the skin level exit site, approximately two to three inches (50.8 mm-76.2 mm), decreases the chances of infection. The tunneling procedure, well known in the art, is also used for insertion of the Hickman or Broviac catheter manufactured by C.R. Bard Inc. (Murray Hill, N.J.).

After exposing the blood vessel and making the adjacent incision for tunneling purposes, the proximal end of sheath 3 is passed into the blood vessel and the tissue is then sutured around the sheath fixing it in place. It is preferred that the infection barrier sleeve 4 be disposed about the entire length of the portion of the sheath 3 residing in the subcutaneous tissue. Catheter 2 is then passed through sheath 3 into the blood vessel to a position appropriate for pumping.

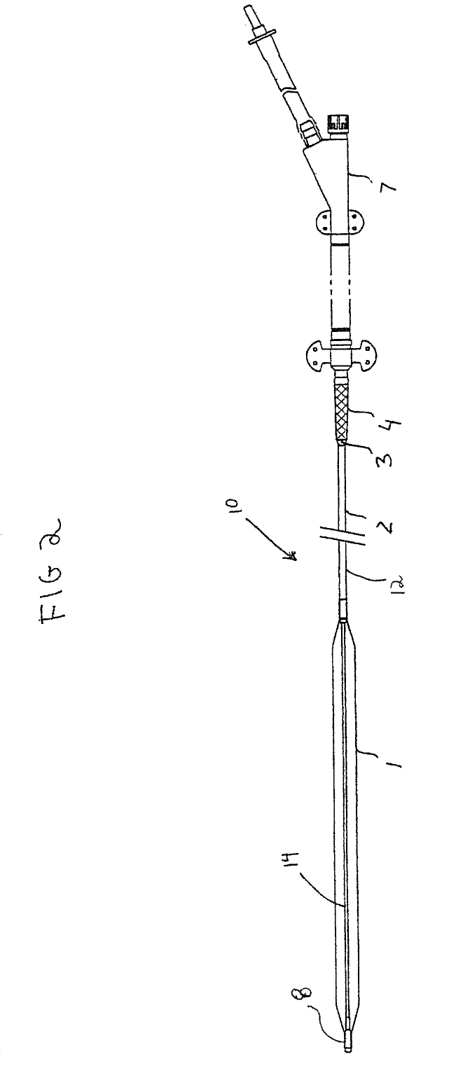

FIG. 2 is a plan view of a long-term dual lumen IAB catheter system 10, as illustrated in FIG. 1, having a mini-sheath 3 which also has an infection barrier sleeve 4 disposed about it. The IAB catheter system 10 may be inserted in the same manner as the dual lumen catheter 2 in FIG. 1 except that mini-sheath 3 resides only in the subcutaneous tissue and, thus, does not enter or contact blood vessel 42. Mini-sheath 3 may also be used in the same manner with a single lumen catheter 2 in FIG. 6.

Generally, sheaths serve three major purposes: (i) to control bleeding at the insertion point of the catheter; (ii) to guide the catheter into the subcutaneous tissue; and (iii) to guide the catheter into the artery. Design of mini-sheath 3 serves purposes (i) and (ii) but gives up purpose (iii) in order to reduce the risk of infection to the patient. Having a sheath extend through the subcutaneous tissue and up to or into the artery provides a pathway for bacteria to enter the artery. Use of mini-sheath 3, which resides only in the subcutaneous tissue, therefore, reduces the risk that bacteria will travel down the length of the sheath and enter the vascular system. For an average size patient the length of mini-sheath 3 should be approximately 2 inches (50.8 mm).

The IAB catheter system 10, illustrated in FIGS. 1, 2 and 6, is designed for long-term use. This involves the incorporation of infection barrier sleeve 4, as discussed above, and also optional implantation in the upper body of a patient. The IAB catheter is appropriately sized for such long term implantation in the smaller arteries. The length of the catheter 2 should be between 7 and 13 inches. Preferably, the distal end of the Y-fitting 7 and the proximal end of the membrane should be 10 inches apart. Note that the standard IAB catheter length is 20 inches. The diameter of the catheter 2 and the balloon membrane 1 in a wrapped state is preferably less than approximately 8 Fr. To avoid compromising upper limb circulation a diameter of 6 Fr. is more preferable.

What is claimed is:

1. A method for inserting a balloon catheter into a patient, said catheter comprising a balloon membrane, an outer tube and a tip, said balloon membrane connected on one end to the tip and on an opposite end to the outer tube, said method comprising the steps of:
(a)creating a single incision in the skin of the patient;
(b)inserting a guide wire through said incision through subcutaneous tissue and into a blood vessel of a patient;
(c)passing a sheath over said guide wire into said patient such that one end of the sheath is in the blood vessel and a opposite end of the sheath remains outside the patient and such that an infection barrier sleeve disposed on said sheath resides solely in subcutaneous tissue and does not enter or contact the blood vessel;
(d)passing the balloon catheter over the guide wire through the sheath and at least partially into the blood vessel;
(e)advancing said balloon catheter to a position in the vasculature appropriate for therapy.

2. The method as claimed in claim 1 wherein the balloon catheter further comprises an inner tube disposed within an outer surface of the outer tube and wherein in step (d) the balloon catheter is passed over the guide wire by passing the guide wire through the inner tube.

3. The method as claimed in claim 1 wherein the tip further comprises a tip lumen and wherein in step (d) the balloon catheter is passed over the guide wire by passing the guide wire through said tip lumen.

4. The method as claimed in claim 1 wherein the tip further comprises a tip lumen and balloon catheter further comprises a stylet connected on one end to the tip and on an opposite end to the outer tube and wherein in step (d) the balloon catheter is passed over the guide wire by passing the guide wire through said tip lumen.

5. The method as claimed in claim 1 further comprising the steps of inserting an angiographic needle into the blood vessel after the incision in step (a) and then in step (b) passing the guide wire through said angiographic needle into the blood vessel.

6. The method as claimed in claim 2 further comprising the steps of inserting an angiographic needle into the blood vessel after the incision in step (a) and then in step (b) passing the guide wire through said angiographic needle into the blood vessel.

7. The method as claimed in claim 3 further comprising the steps of inserting an angiographic needle into the blood vessel after the incision in step (a) and then in step (b) passing the guide wire through said angiographic needle into the blood vessel.

8. The method as claimed in claim 4 further comprising the steps of inserting an angiographic needle into the blood vessel after the incision in step (a) and then in step (b) passing the guide wire through said angiographic needle into the blood vessel.

9. The method as claimed in claim 1 wherein the infection barrier sleeve is made from felt.

10. The method as claimed in claim 2 wherein the infection barrier sleeve is made from felt.

11. The method as claimed in claim 3 wherein the infection barrier sleeve is made from felt.

12. The method as claimed in claim 4 wherein the infection barrier sleeve is made from felt.

13. The method as claimed in claim 5 wherein the infection barrier sleeve is made from felt.

14. The method as claimed in claim 1 further comprising the step of passing a dilator over the guide wire prior to passing the sheath over the guide wire.

15. The method as claimed in claim 1 wherein the balloon catheter is an intra-aortic balloon catheter and further comprising the step of repeatedly inflating and deflating the balloon membrane after final positioning of said balloon catheter in step (e).

16. The method as claimed in claim 1 wherein the sheath has a length no longer than 3 inches (76.2 mm).

17. The method as claimed in claim 2 wherein the sheath has a length no longer than 3 inches (76.2 mm).

18. The method as claimed in claim 3 wherein the sheath has a length no longer than 3 inches (76.2 mm).

19. The method as claimed in claim 4 wherein the sheath has a length no longer than 3 inches (76.2 mm).

20. The method as claimed in claim 5 wherein the sheath has a length no longer than 3 inches (76.2 mm).

21. The method as claimed in claim 1 wherein the sheath has a length no longer than 3 inches (76.2 mm) and wherein the sheath is made from felt.
